(19) United States
(12) Patent Application Publication
(10) Pub. No.: US 2020/0315146 A1
(43) Pub. Date: Oct. 8, 2020

(54) APPLICATION OF PLURIPOTENT STEM CELLS HAVING MODIFIED DIFFERENTIAL POTENTIAL TO PRODUCING ANIMALS

(71) Applicant: The University of Tokyo, Bunkyo-ku (JP)

(72) Inventors: Hiromitsu NAKAUCHI, Tokyo (JP); Hideyuki SATO, Tokyo (JP); Hideki MASAKI, Tokyo (JP); Motoo WATANABE, Tokyo (JP)

(73) Assignee: The University of Tokyo, Bunkyo-ku (JP)

(21) Appl. No.: 16/652,803

(22) PCT Filed: Oct. 9, 2018
(86) PCT No.: PCT/JP2018/037566

§ 371 Date: Jun. 16, 2020

(30) Foreign Application Priority Data
Oct. 10, 2017 (JP) 2017-197034

(51) Int. Cl. A01K 67/027; C12N 5/0735; C12N 5/10

(52) U.S. Cl. CPC A01K 67/0271; C12N 15/85; C12N 5/10; C12N 5/0606

(57) ABSTRACT

The present invention provides a method for preparing a somatic chimera by using a pluripotent cell genetically engineered not to differentiate into a predetermined type of cell. The present invention particularly provides a method for preventing a pluripotent cell from contributing to the brain and gonad in a somatic chimera animal.

### TECHNICAL FIELD

The present invention relates to application of a pluripotent stem cell having a modified differentiation potential to preparation of an animal.

### BACKGROUND ART

Genetically modified animals are frequently used for analyzing in-vivo gene function; transgenic animals having a foreign gene introduced therein and gene-knockout animals having an endogenous gene disrupted are known.

A genetically modified animal is produced by introducing a pluripotent cell having a desired gene modification into a wild-type animal embryo or by introducing a desired gene modification directly into an animal embryo. When an animal embryo having a pluripotent cell having a desired genetic modification introduced therein is transplanted in the uterus of a false pregnant foster mother and allowed to develop, the individual develops from the animal embryo has becomes a chimeric state consisting of wild-type cells and genetically modified cells.

Non Patent Literature 1 discloses that a Prdm14 gene knockout mouse lacks a germ cell. Since the Prdm14 gene knockout mouse lacks the germ cell, it cannot be crossed.

### CITATION LIST

### Non Patent Literature

Non Patent Literature 1: Yamaji, M. et al., Nature Genetics, 40(8): 1016-1022, 2008

### SUMMARY OF INVENTION

The present invention provides application of a pluripotent stem cell having a modified differentiation potential to preparation of an animal.

In the present invention, the present inventors propose that a pluripotent cell having a modified differentiation potential is introduced into an animal embryo and the animal embryo is allowed to develop to obtain an animal, and the distribution of the pluripotent cell in the animal body is controlled.

The present inventors more specifically propose that a genetically engineered cell (for example, knockout cell, knockdown cell or a transgenic cell) is introduced into an embryo, thereby inhibiting or losing the differentiation ability of the cell into a target tissue (tissues other than the target tissue may be differentiated in accordance with chimera-forming ability that the genetically engineered cell inherently has).

In this manner, the present inventors propose that a chimeric animal is created having a target tissue consisting essentially of a cell derived from an embryo and the other tissues which are chimeras between a cell derived from the embryo and a cell derived from the cell introduced into the embryo.

The present invention has been attained based on the aforementioned technical idea. The present invention is provided as follows:

(1) A method for preparing a somatic chimera animal, comprising
introducing a pluripotent cell into an embryo to obtain a somatic chimera animal, wherein
the pluripotent cell is genetically engineered not to differentiate into a predetermined type of cell.

(2) A composition for preparing a somatic chimera animal, comprising a pluripotent cell, wherein the pluripotent cell is genetically engineered not to differentiate into a predetermined type of cell.

(3) The method according to (1), wherein the pluripotent cell genetically engineered not to differentiate into a predetermined type of cell is a cell in which a gene essential for a germ cell is disrupted.

(4) The composition according to (2), wherein the pluripotent cell genetically engineered not to differentiate into a predetermined type of cell is a cell in which a gene essential for a germ cell is disrupted.

(5) The method according to (1) or (3), wherein the pluripotent cell genetically engineered not to differentiate into a predetermined type of cell is a Prdm14 gene knockout cell, an Otx2 gene knockout cell or a Prdm14 gene/Otx2 gene double knockout cell.

(6) The composition according to (2) or (4), wherein the pluripotent cell genetically engineered not to differentiate into a predetermined type of cell is a Prdm14 gene knockout cell, an Otx2 gene knockout cell or a Prdm14 gene/Otx2 gene double knockout cell.

(7) The method according to (3) or (5), wherein the somatic chimera animal and the pluripotent cell are female.

(8) The method according to (3) or (5), wherein the somatic chimera animal and the pluripotent cell are male.

(9) Use of a pluripotent cell in production of a somatic chimera animal, wherein the pluripotent cell is genetically engineered not to differentiate into a predetermined type of cell.

(10) A non-human somatic chimera animal, wherein a predetermined type of cell consists of a cell derived from a host animal.

(11) The non-human somatic chimera animal according to (10), wherein a germ cell consists of the cell derived from a host animal.

The present invention is useful in preparing a somatic chimera animal having a controlled chimera. Preparation of such a somatic chimera animal can be advantageously used in analyzing information network between an introduced cell and a host animal.

### BRIEF DESCRIPTION OF DRAWINGS

FIG. 1 shows a chimera rate of a cell introduced in an embryo in a somatic chimera animal, which was obtained by introducing a wild type, male or female Prdm14 gene knockout ES cell into a wild-type embryo.

FIG. 2 shows a method for disrupting Prdm14 gene by CRISPR/Cas9 system, and shows Prdm14 gene disrupted.

FIG. 3A shows a cell-specific cell death induction system.

FIG. 3B shows a cell-specific cell death induction system.

FIG. 3C shows a cell-specific cell death induction system.

FIG. 3D shows a cell-specific cell death induction system.

FIG. 4 shows a method for preparing an Otx2 knockout ES cell.

FIG. 5 shows the overall distribution of a transplanted cell in an individual obtained by introducing an Otx2 knockout ES cell into a wild-type embryo, and shows the results of immunochemical staining of brain-tissue sections.

FIG. 6 shows expression of GFP in the pancreas of an individual (#11), which was obtained by introducing Prdm14 knockout (Prdm14−/−) and Otx2 knockout (Otx2−/−) GFP expressing ES cells into a Pdx1−/−embryo.

FIG. 7 shows that the pancreas of an individual (#11) has a normal function by a glucose tolerance test; and a genotype of Pdx1 gene of a fetus obtained in a crossing test of an individual (#11) and a wild-type mouse.

### DESCRIPTION OF EMBODIMENTS

As used herein, the “germ cell” refers to a cell species differentiated from a primordial germ cell as a specific cell population forming a germ cell; more specifically, refers to all cell species in the germline generated during a differentiation process from a primordial germ cell to gametes such as a sperm and an egg.

As used herein, the “animal defective in own germ cell in the developmental stage” or an embryo thereof refers to an animal which does not generate its own germ cell in the developmental stage or an embryo thereof. Examples of such an animal include an animal having a cell-autonomous defect, that is, an animal defective in own germ cell in the developmental stage.

As used herein, the “differentiation potential” used for a pluripotent cell refers to a potential of the pluripotent cell to differentiate into tissues present in a fetus and an adult.

As used herein, the “cell-autonomous defect” means that abnormality intrinsic to a cell substantially and quantitatively or qualitatively affects only to the cell. Preferably, the “cell autonomous defect” is not a defect affecting other cells. Since the “cell autonomous defect” is a defect having an effect only on the cell having the defect, if a non-defective cell is introduced into a host, the cell does not receive any effect from the host. Examples of the “cell autonomous defect” include modification inducing tissue-specific cell death and modification depriving a cell autonomous factor required for tissue differentiation.

As used herein, the “animal” refers to a mammal and a bird. Examples of the animal include, but are not particularly limited to, a rodent such as a mouse and a rat, a livestock animal such as a pig and a cow, a pet animal such as a dog and a cat, a bird such as a chicken, and a primate such as a monkey. As used herein, the animal is a non-human animal.

As used herein, the “intraspecies” refers to isogenic and allogenic. The “interspecies” refers to different species in the animal taxonomy.

As used herein, the “somatic chimera animal” refers to an animal having a tissue or organ in which the cell of an individual coexist (at the cell level) with the cell of another individual (for example, isogenic cells or isogenic cells having one or more genetic modifications or allogeneic cells). In the present invention, a somatic chimera animal can be obtained by introducing a plurality of pluripotent cells (for example, about 10 cells) into an embryo (for example, morula and blastocyst). More specifically, as the embryo, embryos in various stages from 8 cell-stage to a blastocyst stage can be used. A method for introducing pluripotent cells into an animal embryo is known to those skilled in the art. In introducing the cells into an embryo in a blastocyst stage, the cells may be introduced, for example, in the cleavage cavity. If an early-stage embryo up to the morula stage is used, the cells may be assembled by allowing them into contact to each other. Examples of the pluripotent cell that can be introduced into an embryo in the present invention include a pluripotent stem cell such as an ES cell and an iPS cell, and a pluripotent cell such as inner cell mass (ICM). The number of the pluripotent stem cells to be introduced into an embryo can be appropriately determined and are not particularly limited; for example, about 3 to 10 cells, can be introduced into an embryo.

As used herein, the “host animal” refers to an embryo to which pluripotent cells are to be introduced in producing a somatic chimera animal.

As used herein, “the cell to be introduced into an embryo” refers to the cell to be introduced into an embryo in producing a somatic chimera animal.

As used herein, the “pluripotent cell” refers to a pluripotent stem cell such as an ES cell and an iPS cell, and a pluripotent cell such as inner cell mass (ICM). The pluripotent cell is known to be differentiated into any types of cells of a fetus or an adult.

As used herein, “genetic engineering” refers to modification of gene expression and genetic modification.

As used herein, modification of gene expression refers to modification by which gene expression is enhanced or attenuated. Gene expression can be enhanced by connecting an endogenous gene operatively to at least one regulatory sequence to allow the endogenous gene to strongly express within the cell. Gene expression can be attenuated by a knockdown technique using an antisense nucleic acid to an endogenous gene, siRNA and a nucleic acid derivative thereof (for example, bridged nucleic acid (BNA), locked nucleic acid (LNA), a hybrid nucleic acid containing different types of nucleic acids, a peptide nucleic acid (PNA)) and shRNA and a nucleic acid derivative thereof.

As used herein, “genetic modification” means that a gene is different from a wild type gene and includes a naturally occurring modification and an artificial modification. Representative examples of the genetic modification include transgenic modification and knockout.

As used herein, “crossing” refers to fertilization between two individuals in order to obtain a next generation; in other words, obtaining a fetus from two individuals as parents. Examples of crossing include artificial crossing by an insemination technique and pairing of a male and a female for reproduction.

The present invention provides a method for preparing a somatic chimera animal, comprising introducing a pluripotent cell having a modified differentiation potential into an embryo.

In an embodiment of the present invention, examples of the pluripotent cell having a modified differentiation potential include a pluripotent cell genetically engineered not to differentiate into a predetermined type of cell. Therefore, the present invention provides a somatic chimera animal, in which the predetermined type of cell consists essentially of the cell derived from an embryo; whereas the other cells consist of the cell derived from the embryo and a cell derived from the cell introduced into the embryo (depending on the chimera formation ability of the cell introduced into the embryo) and provides a method for producing such an animal.

In an embodiment of the present invention, preferably, the pluripotent cell having a modified differentiation potential may have a cell autonomous defect. For example, a pluripotent cell, which has a cytotoxic gene operatively connected to an enhancer and/or a promoter of a gene that is induced when the cell differentiates into a predetermined type of cell, autonomously dies when it differentiates into the predetermined type of cell. Thus, such a pluripotent cell can be employed as the pluripotent cell having an autonomous defect and a modified differentiation potential.

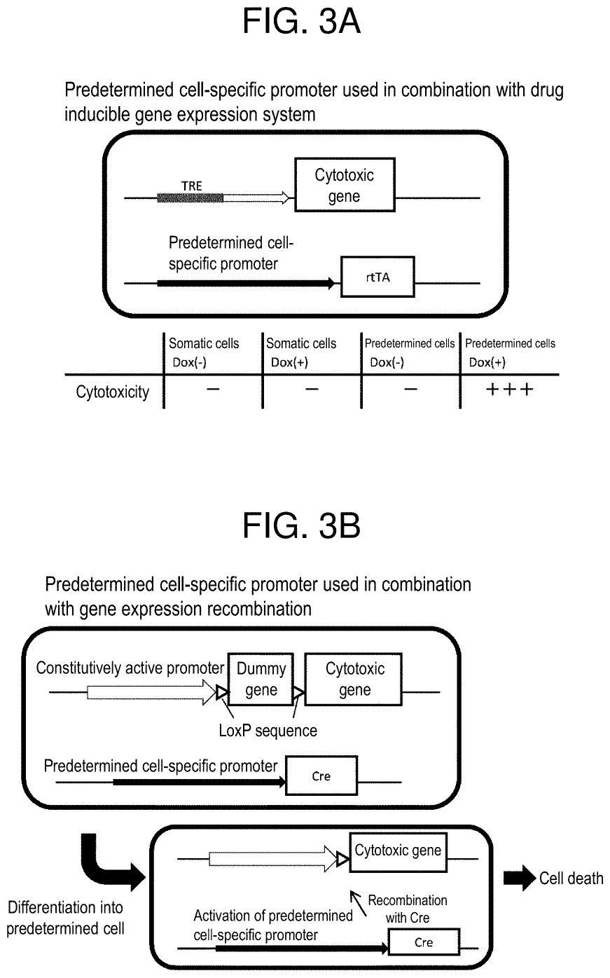

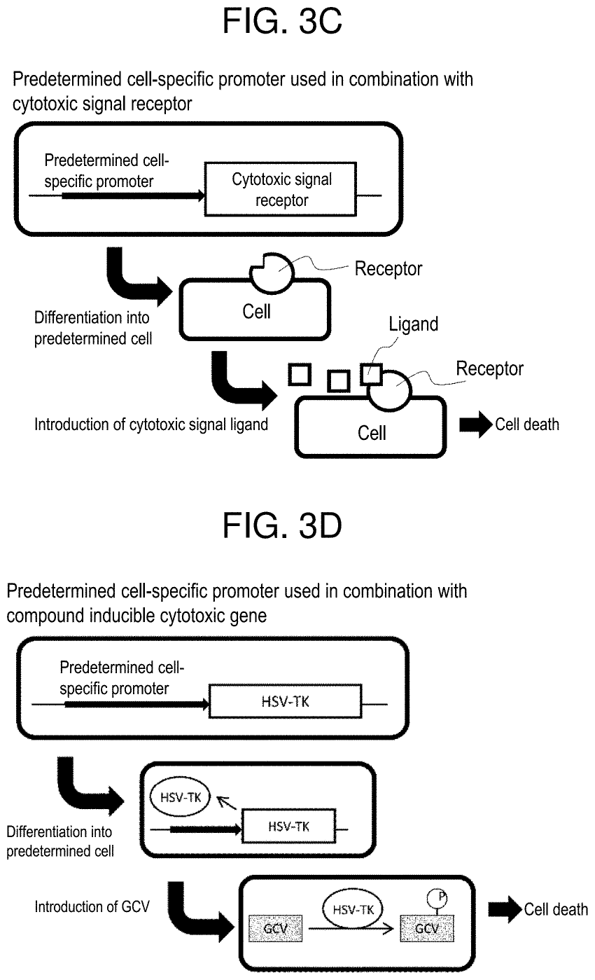

In an embodiment of the present invention, cell autonomous cell-death can be induced. Cell death can be induced by expressing a cytotoxic gene such as Caspase-8, Caspase-9, Barnase, and diphtheria toxin in a predetermined cell (such as a germ cell) depending on the concentration of a drug (for example, doxycycline) within an embryo, by using, for example, a drug-inducible gene expression system (for example, tetracycline-responsive expression induction system) in combination. Now, a method for producing cell-specific cytotoxicity will be described below with reference to FIGS. 3A to D.

FIG. 3A shows a system for inducing cell-specific and drug-concentration dependent cell-death by using a cell-specific promoter in combination with a drug-inducible gene expression system. The drug-inducible gene expression system herein is Tet-On system. In FIG. 3A, a reverse tetracycline-regulated transactivator (rtTA) is operatively connected to a cell-specific promoter. A fetus to which this system is integrated, expresses rtTA in a cell-specific manner. Herein, a cytotoxic gene is induced only in a cell expressing rtTA by a tetracycline compound such as doxycycline (Dox). In this way, cell-specific cell death can be induced.

Another example of the methods for attaining the same purpose is a method for expressing a cytotoxic gene in a cell-specific manner by a genetic recombination system such as Cre-LoxP. FIG. 3B shows a system for inducing cell death in a cell-specific manner by using a tissue-specific gene recombination system such as Cre-LoxP. In FIG. 3B, a physiological meaningless gene (dummy gene) is connected to a cytotoxic gene at the downstream of a constitutively active promoter. In this state, cell death cannot be induced in the cell. However, in this case, a LoxP sequence is each arranged at the upstream and downstream of the dummy gene. In addition, this cell has Cre operatively connected to a cell-specific promoter. In this system, when the cell is differentiated into a predetermined cell, Cre is driven by the cell-specific promoter and expressed in a cell-specific manner, and acts on the LoxP sequences to remove the dummy gene. In this manner, the cytotoxic gene is allowed to connect operatively to a constitutively activated promoter to express the cytotoxic gene in a cell-specific manner, with the result that the cell can be induced in a cell-specific manner. In this system, Cre recombinase may be replaced by CreER, which is activated by 4-tamoxifen hydroxide. CreER is known as a fusion protein of Cre recombinase and a mutant of a ligand binding region in estrogen receptor, which is activated by 4-tamoxifen hydroxide.

Still another example of the methods for attaining the same purpose is a method of inducing cell death in a cell-specific manner by expressing a cytotoxic-signal receptor in a cell-specific manner and allowing a ligand to act on the receptor at appropriate timing. In this case, the cell that does not express a cytotoxic signal receptor is preferably insensitive to the ligand. In FIG. 3C, a system, in which a cytotoxic-signal receptor is operatively connected to a cell-specific promoter, is integrated into a cell. When the cell is differentiated into a predetermined cell, the cytotoxic signal receptor is expressed on the cell surface and cell death is induced in a ligand-dependent manner. Examples of the cytotoxic-signal receptor and a ligand thereof include a diphtheria toxin receptor and diphtheria toxin.

Yet another example of the methods for attaining the same purpose is a compound inducible cytotoxic system. Examples of other compound inducible cytotoxic system include HSV-TK/GCV system (Moolten F L et al., Hum. Gene Ther. 1990; 1: 125-134) and a system using inducible caspase-9 (Straathof K C et al., Blood 2005; 105: 4247-4254).

FIG. 3D shows the HSV-TK/GCV system. To describe it more specifically, ganciclovir (GCV) not phosphorylated has a weak cytotoxicity; however, if a thymidine kinase gene (HSV-TK) that the genus herpesvirus has, is allowed to act on GCV, GCV is phosphorylated and converted into GCV triphosphate to acquire cytotoxicity. Thus, if HSV-TK is operatively connected to a cell-specific promoter and expressed in a cell-specific manner, cell death can be induced in a cell-specific manner. Inducible caspase-9 is a protein obtained by replacing CARD in caspase-9 with FKBP12, and can be dimerized and activated only in the presence of a tacrolimus derivative such as AP1903 to induce cell death. If the system, in which inducible caspase-9 is operatively connected to a cell-specific promoter, is introduced into a cell and the cell is differentiated into a predetermined cell, cell death is induced by the tacrolimus derivative in a cell-specific manner.

In the present invention, the aforementioned methods can be used in combination. Induction of cell-specific cell death can be attained by those skilled in the art based on the contents of the specification and the technical knowledge in the art.

In an embodiment of the present invention, for example, a cell autonomous defect can be generated in a germ cell. For example, a pluripotent cell, which has a cytotoxic gene operatively connected to an enhancer and/or a promoter of a gene expressed specifically in a germ-cell, such as Prdm14, Nanos2, Nanos3, DDX4, and Sox17, autonomously die if it is differentiated into a germ cell. Because of this, such a pluripotent cell does not contribute to a germ cell of a somatic chimera animal. In an embodiment of the present invention, the gene specifically expressed in a germ cell can be, for example, a gene temporarily or long and specifically expressed in a germ cell.

In an embodiment of the present invention, for example, contribution of a pluripotent cell to the cerebral cortex can be limited. For example, a pluripotent cell, which has a cytotoxic gene operatively connected to an enhancer and/or promoter of a gene specifically expressed in a forebrain (the cerebrum in future), such as Otx1 and Otx2, and which is destined to develop into the forebrain dies. Thus the pluripotent cell does not contribute to the cerebral cortex in a somatic chimera animal. In an embodiment of the present invention, a gene specifically expressed in the forebrain can be defined as a gene temporarily and long; and substantially expressed in the forebrain region. Then, disadvantage brought by contribution of the cell introduced into an embryo to the brain, for example, occurrence of a damage on a higher brain function can be avoided.

In an embodiment, even if Izumo gene specifically expressed in a sperm is deleted, sperms can be formed; however it is known that the sperms formed do not have a fertilization function. Deletion of such a gene may induce dysfunction of gametes.

In an embodiment, a gamete may be removed by using a sperm-specific promoter/enhancer such as Izumo's one in combination with a cytotoxic gene. Similarly, using a promotor and/or enhancer of a gene such as Tpap, Acrosin and Tra98 specifically expressed in, for example, a sperm and a gene such as Gdf9, Zp1 and Zp3, specifically expressed in an egg in combination with a cytotoxic gene, a gamete can be removed in the gamete formation process.

In another embodiment, formation of a germ cell can be avoided by using a promoter/enhancer of a gene such as Dazl, Stra8, Taf7l or Sycp3, involved in the meiosis that specifically occurs in a germ cell.

In an embodiment of the present invention, as the cell to be introduced into an embryo, a cell in which a gene essential for germ cell differentiation is disrupted, can be used.

Prdm14 is a gene encoding a PR domain-containing protein 14. Prdm14 is also referred to as PR domain zinc finger protein 14. Prdm14 protein has an amino acid sequence registered, for example, under HPRD ID: 11457 and the gene thereof is present at position 8q13.3 on a chromosome. In a Prdm14 gene-knockout mouse, it is known that the germ cells in the ovary and testis are deleted (Yamaji M. et al., Nature Genetics, 40: 1016-1022, 2008).

In an embodiment of the present invention, a pluripotent cell, from which Prdm14 gene is knocked out or knocked down to prevent differentiated into a germ cell, may be used as the cell to be introduced into an embryo. In an embodiment of the present invention, Prdm14 gene can be knocked out by, e.g., deleting a part or whole of the gene or introducing a frame shift. In an embodiment of the present invention, knockout Prdm14 gene has the sequence represented by SEQ ID NO:3 or SEQ ID NO:4.

If a somatic chimera animal is prepared by introducing a pluripotent cell genetically engineered not to be differentiated into a predetermined type of cell as described above, into an embryo as the cell to be introduced into an embryo, the cells except the predetermined type of cell form a chimera depending on the chimera formation ability intrinsic to the cell introduced into an embryo. As a result, a somatic chimera animal, in which a predetermined type of cell consists essentially of an embryo-derived cell, can be obtained. According to the present invention, as described above, it is possible to obtain a somatic chimera animal, in which the tissues except the target tissue have a chimera depending on the chimera formation ability intrinsic to the cell introduced into an embryo; whereas the target tissue consists essentially of the embryo-derived cell.

According to the present invention, as the pluripotent cell genetically engineered not to be differentiated into a predetermined type of cell, a Prdm14 knockout and Otx2 knockout pluripotent cell (for example, ES cell and iPS cell) can be used. As described, the present invention provides an invention using a pluripotent cell genetically engineered not to differentiate into the brain and gonad as the pluripotent cell genetically engineered not to be differentiated into a predetermined type of cell.

Interestingly, as is apparent from the following Examples, in the living body, the cell introduced into an embryo and a host cell have high flexibility, that is, even if one of the cells cannot be differentiated into a predetermined tissue, the other cell complementarily is differentiated into the tissue. To be more specific, if the cell introduced into an embryo is not differentiated into a predetermined type of cell, the embryo cell of a host can be complementarily differentiated, with the result that the predetermined type of cell (differentiated from the cell introduced into an embryo) forms a chimera consisting of the cell introduced into an embryo and the cell of a host embryo. In contrast, if the cell introduced into an embryo is not differentiated into a predetermined type of cell, the cell derived from a host embryo consists the predetermined type of cell. A differentiation defect of the cell introduced into an embryo is complemented by the host embryo cell, with the result that the defect will not appear as an abnormality in the somatic chimera animal.

It has been demonstrated that, in blastocyst complementation of an organ-deficient animal, a defective organ is complemented by an organ consisting of the cell introduced into an embryo (for example, WO2010/087459, which is incorporated by citation as a part of the specification of the present application).

Accordingly, in an embodiment, as a pluripotent cell genetically engineered not to be differentiated into a predetermined type of cell, a Prdm14 knockout and Otx2 knockout pluripotent cell (for example, ES cell and iPS cell) can be used; and, as the embryo, a Pdx1−/− knockout embryo or a Pdx1-Hes1 transgenic embryo can be used. In an embodiment, as the pluripotent cell genetically engineered not to be differentiated into a predetermined type of cell, a Prdm14 knockout and Otx2 knockout pluripotent cell (for example, ES cells and iPS cell) can be used; and, as the embryo, an Sall1 knockout embryo can be used.

According to the present invention, as the pluripotent cell genetically engineered not to be differentiated into a predetermined type of cell, a pluripotent cell genetically engineered not to be differentiated into the brain and gonad can be used. The pluripotent cell genetically engineered not to be differentiated into the brain and gonad can be prepared by genetic engineering not to differentiate into the brain and genetic engineering not to differentiate into the gonad.

For example,
a pluripotent cell having both
a cytotoxic gene, which is operatively connected to an enhancer and/or promoter of a gene exhibiting germ cell specific expression, such as Prdm14, Nanos2, Nanos3, DDX4, and Sox17, and
a cytotoxic gene, which is operatively connected to an enhancer and/or promoter of a gene exhibiting forebrain (the cerebrum in future) specific expression, such as Otx1 and Otx2,
can be used as the pluripotent cell genetically engineered not to be differentiated into a predetermined type of cell.

For example,
a pluripotent cell having both
a cytotoxic gene operatively connected to an enhancer and/or promoter of a gene exhibiting gamete-specific expression, for example, a gene exhibiting a sperm-specific expression, such as Izumo, Tpap, Acrosin, or Tra98, or an egg-specifically expressing gene such as Gdf9, Zp1 or Zp3; and
a cytotoxic gene operatively connected to an enhancer and/or promoter of a gene exhibiting forebrain (the cerebrum in future) specific expression, such as Otx1 and Otx2,
can be used as the pluripotent cell genetically engineered not to be differentiated into a predetermined type of cell.

For example,
a pluripotent cell having both
a cytotoxic gene operatively connected to an enhancer and/or promoter of a gene involved in meiosis, which is a gamete-specific phenomenon, such as Dazl, Stra8, Taf7l or Sycp3; and
a cytotoxic gene operatively connected to an enhancer and/or promoter of a gene exhibiting a forebrain (the cerebrum in future) specific expression, such as Otx1 and Otx2,
can be used as the pluripotent cell genetically engineered not to be differentiated into a predetermined type of cell.

As described above, a predetermined type of cell alone in an individual obtained can be derived from the cell introduced into an embryo, or only a predetermined type of cell in an individual obtained can be derived from embryo cell of a host. Accordingly, the present invention provides a technique freely controlling only a predetermined tissue to consist of genetically engineered cells, controlling only a predetermined tissue to consist of wild type cells, or controlling only a predetermined tissue to have a chimera; and provides a technique for analyzing the function of the gene engineered in a predetermined tissue by mutually comparing these. As a result, the technique for controlling the chimera of a somatic chimera animal provided by the present invention, gives a new choice to a chimera control technology so far used.

Thus, according to an embodiment of the present invention, there is provided a method for preparing a somatic chimera animal, comprising introducing a pluripotent cell into an embryo to obtain a somatic chimera animal, wherein the a pluripotent cell is genetically engineered not to differentiate into a predetermined type of cell, and the embryo is genetically engineered to delete a predetermined type of cell different from the pluripotent cell. Examples of the genetic engineering of an embryo include knockout of Pdx1 gene or deletion of the pancreas by a Pdx1-Hes1 transgenic technique and deletion of the kidney by knockout of Sal1 gene.

In an embodiment of the present invention, the genetic engineering for deleting a predetermined type of cell in an embryo, includes loading a cytotoxic gene operatively connected to a promoter and/or enhancer specific to a predetermined type of cell. In some embodiments, an embryo may be genetically engineered by genetic modification (for example, knockout or transgenic technique), which are known to delete a predetermined cell or tissue. Accordingly, in an embodiment of the present invention, an embryo has a cytotoxic gene operatively connected to a promoter and/or enhancer specific to a predetermined cell or may have a gene modified such that a predetermined cell or tissue is deleted.

In an embodiment of the present invention, examples of the embryo to which a cell is to be introduced, include a vertebrate-derived embryo such as an embryo derived from a non-human mammal including an embryo derived from a non-human primate such as chimpanzee, gorilla, orangutan, monkey, marmosets and bonobo; an embryo derived from a non-human mammal such as pig, rat, mouse, cow, sheep, goat, horse and dog (for example, embryo of Carnivora, artiodactyla, perissodactyla and rodent); and an embryo of a bird such as a chicken.

In an embodiment of the present invention, examples of the cell to be introduced into an embryo include, but are not particularly limited to, a pluripotent cell of a primate such as a human and a monkey, and, a pluripotent cell of a mammal such as pig, cow, sheep and goat. In an embodiment of the present invention, the cell to be introduced into an embryo is a human pluripotent cell (for example, ES cell or iPS cell).

In the present invention, an embryo and the cell to be introduced into the embryo may be intraspecies or interspecies to each other (see, for example, WO2010/087459, which is incorporated by citation as a part of the specification of the present application). In the present invention, combinations of an embryo and the cell to be introduced into an embryo includes a combination of a mouse and a rat. A chimeric animal was successfully produced by blastocyst complementation between a mouse and a rat (see, WO2010/021390 and WO2010/087459, which are incorporated by citation as parts of the specification of the present application). The genetic distance between a mouse and a rat is equivalent to a human-pig genetic distance. Thus, successful preparation of a interspecies chimeric animal between a mouse and a rat, means that a interspecies chimeric animal can be sufficiently produced if the genetic distance between the species closer than the human-pig genetic distance. In WO2014119627, a somatic chimera animal between a human and a mouse or between a marmoset and a mouse are obtained. Accordingly, a chimeric animal can be produced even from a rodent-primate combination. An interspecies chimeric animal can be produced when the genetic difference is considered to be smaller than the rodent-primate distance, a chimeric animal can be produced between the two species. From the above, examples of an interspecies combination of an embryo and the cell to be introduced into the embryo used in the present invention other than a combination of a mouse and a rat, include a combination between non-human mammals, a combination between birds, a combination between a human and a non-human primate, a combination between a human and a chicken, a combination between a human and a pig, a combination between human and a cow, a combination between a human and a goat, a combination between a human and a sheep, a combination between non-human primates, a combination between a non-human primate and a pig, a cow or a sheep, a combination between a cow and a pig, a sheep, a goat or a horse, and a combination between a pig and a sheep, goat or a horse. Also, a combination between a human and an animal belonging to any one of a carnivora, artiodactyla, perissodactyla, may be employed. Combinations between animals belonging to the same genus, species or families may be accepted. Although a mouse and a rat differ in the number of chromosome, it is possible to produce a chimeric animal between a mouse and a rat. From this, even if animals differ in the number of chromosomes, the possibility of producing a chimeric animal is not denied.

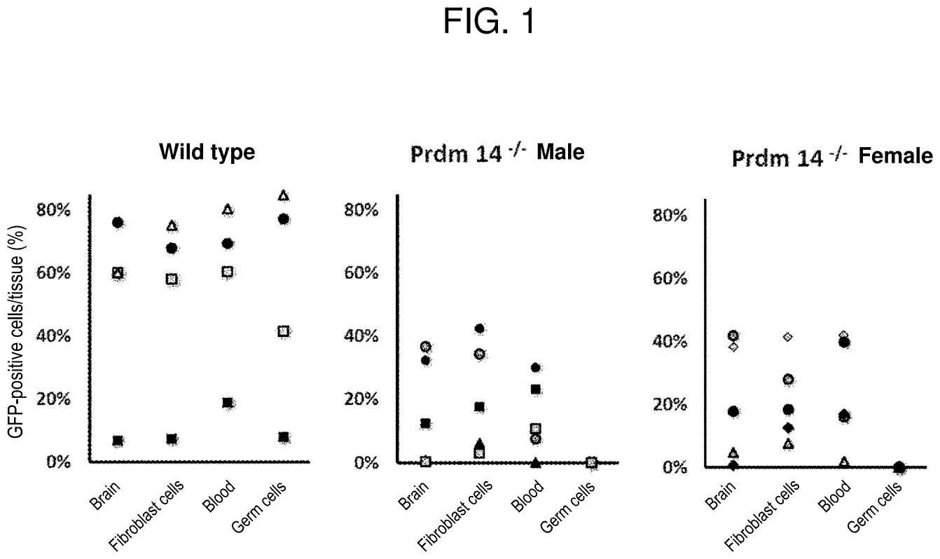

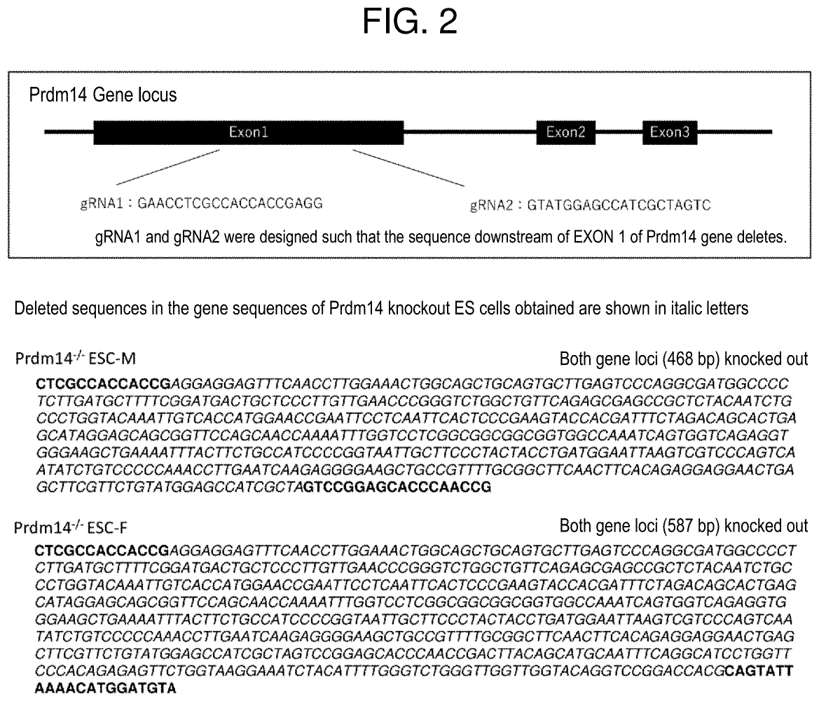

It is considered that the chimera formation ability of a pluripotent cell is different depending on type of cell (see, for example, the panel “wild type” of FIG. 1). Therefore, a pluripotent cell that can provide a desired chimera except a predetermined type of cell can be selected and used. The chimera varies depending on the chimera formation ability of a pluripotent cell. If the chimera formation ability of a pluripotent cell is high, the percentage of the cells derived from the pluripotent cell is high. In contrast, if the chimera formation ability of a pluripotent cell is weak, the percentage of the cells derived from the pluripotent cell is low. The chimera formation ability can be determined by labeling the cell introduced into an embryo and/or an embryo, producing a somatic chimera animal and determining the percentage of the cells labeled in the resultant somatic chimera animal.

Accordingly, in an embodiment, the present invention may include selecting a pluripotent cell having desired chimera formation ability and modifying the differentiation potential of the obtained pluripotent stem cell. The indexes for the chimera formation ability include the strength of the chimera formation ability or the balance in chimera formation ability between tissues is mentioned. Based on the indexes, a pluripotent cell having a desired chimera formation ability may be selected.

In a somatic chimera animal, an embryo to which the cell has been introduced is implanted in the uterus of a false pregnancy female foster parent and allowed to grow into a fetus. A somatic chimera animal may be obtained as a fetus, an offspring or an adult.

According to the present invention, when a pluripotent cell genetically engineered not to differentiate into a germ cell is introduced into an embryo, a germ cell consists essentially of the cell of a host. In this case, if a host animal and the cell to be introduced into an embryo differ in genotype, the germ cell of the resultant somatic chimera animal will have the genotype of the host animal and a fetus or offspring obtained by crossing of a somatic chimera animal will have the genotype of the host animal. This is advantageous in this respect that the genotype of a fetus or offspring obtained by crossing of a somatic chimera animal can be matched with that of the host animal (or, at least the probability thereof is high).

### EXAMPLES

### Example 1: Preparation of Prdm14 Gene Knockout Pluripotent Cell

In this Example, a Prdm14 gene knockout (disrupted) pluripotent cell was produced.

For determining a chimera rate, an embryonic stem cell (ES cell) having EGFP expressed therein was used as a pluripotent cell. Male and female pluripotent cells are both prepared. More specifically, male and female embryos were obtained by crossing a male of an EGFP transgenic mouse and a C57BL6/N female (all purchased from Japan SLC). Then a male ES cell strain (SGE2) and a female ES cell strain (SGE-F13) were established from the male and female embryos, respectively. In this Example, these ES cell strains will be referred to as wild-type ES cells.

Subsequently, a Prdm14 gene knockout (disrupted) ES cell was prepared from a wild-type ES cell. More specifically, a male Prdm14 gene knockout (disrupted) ES cell (Prdm14−/−ESC-M) was prepared by transfecting an SGE2 cell with gRNA cloning vector (Addgene, Plasmid #41824) having Prdm14 gRNA incorporated therein, together with a Cas9 expression vector (Addgene, Plasmid #41815). A female Prdm14 gene knockout (disrupted) ES cell (Prdm14−/− ESC-F) was prepared in the same manner as in the case of Prdm14−/− ESC-M except that SGE-F13 cell was used in place of the SGE2 cell.

### Example 2: Preparation of Chimeric Mouse

In this Example, a chimeric mouse was prepared by using the Prdm14 gene knockout pluripotent cell prepared in Example 1 and a wild-type pluripotent cell.

ICR mice (purchased from Japan SLC) were crossed to obtain an embryo (morula) on Day 2.5. A male or female-derived Prdm14 gene knockout pluripotent cell or a wild-type pluripotent cell was injected into the cavity of the embryo at a rate of 10 cells per embryo, by use of a micromanipulator under a microscope. Thereafter, the embryo was cultured in KSOM-AA (Millipore) for a day and the resultant fertilized egg in the stage of a blastocyst was transplanted into the uterus of a false pregnancy ICR lineage mouse of Day 2.5 after crossing. On Day 12 after transplantation (on Day 14.5 as fetal age), a fetus was taken out by caesarean section. The fetus obtained was observed by a fluorescence microscope and formation of a chimera was determined based on the fluorescence of EGFP as an index.

In individuals (n=4) from which fluorescence emission was observed, the chimera rate of each organ was determined. More specifically, from each of E14.5 chimeric mice from which fluorescence emission was observed, the epidermis (MEF), liver, brain and genital ridge were collected. The MEF, genital ridge and brain were cut into pieces by scissors and thereafter treated with 0.025% trypsin-EDTA (Invitrogen) for 10 minutes at 37° C. to obtain cell suspensions. The liver was disrupted by pipetting to obtain a cell suspension.

To the liver suspension, 0.5 μl of a mouse CD45-APC (eBio) was added. To a genital ridge cell suspension, 0.5 μl of SSEA1-APC (eBio) antibody was added. These were allowed to stand still for 30 minutes on ice in the dark. The liver, MEF, genital ridge and brain were separately washed with phosphate buffered saline (staining medium; SM), and then, resuspended with 1 μg/ml propidium iodide (PI)-containing phosphate buffered saline, and analyzed by FACSAria II (BD Biosciences) and software, Flow-jo. The chimera rate was determined based on the percentage of a GFP positive cell to the whole cells. The results were as shown in FIG. 1.

As shown in FIG. 1, in an individual obtained from the embryo when a wild-type pluripotent cell was introduced into an embryo, a GFP positive cell was observed in a certain percentage in any one of the brain, fibroblast, blood (liver) and a germ cell. In contrast, in the cases where a male-derived Prdm14 gene knockout pluripotent cell and a female-derived Prdm14 gene knockout pluripotent cell were used, a GFP positive cell was not observed in the germ cell. From this, it was demonstrated that a Prdm14 gene knockout pluripotent cell contributes to neither the male-derived germ cell and female-derived germ cell.

### Example 3: Formation of Chimera Using Otx2 Gene Knockout Pluripotent Cell

In this Example, an Otx2 gene knockout pluripotent stem cell was introduced into an embryo.

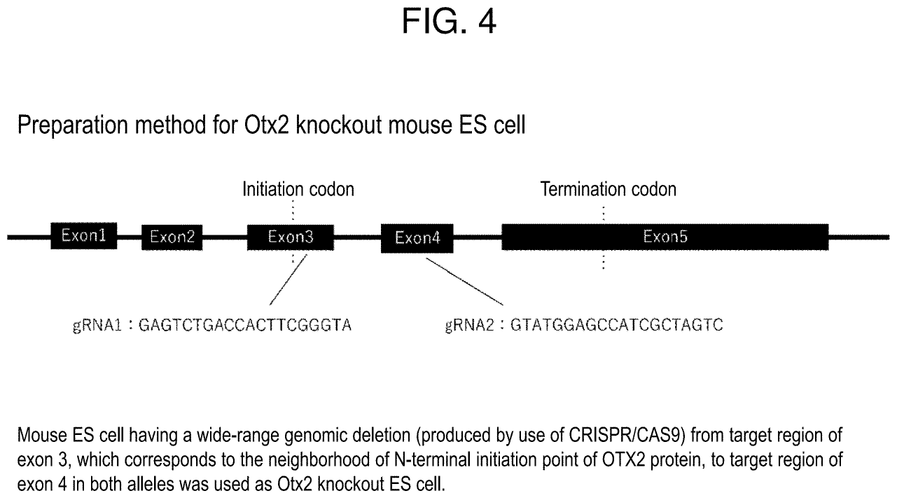

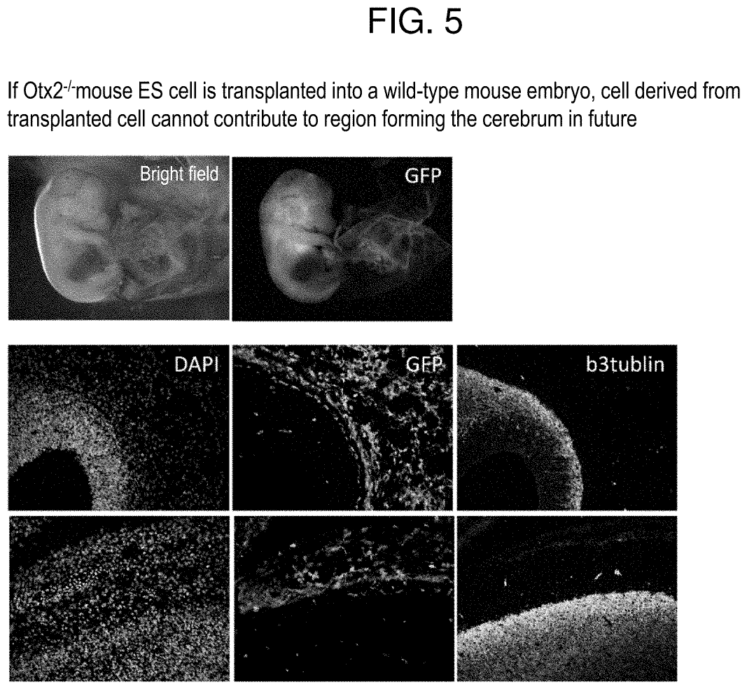

The Otx2 locus of a GFP-expressing mouse ES cell was widely disrupted by use of CRISPR/CAS9. More specifically, a complex of crRNA and tracrRNA, which respectively contain gRNA1 sequence and gRNA2 sequence (shown in FIG. 4), and CAS9 protein (all using Alt-R CRISPR-Cas9 system of Integrated DNA Technologies, Inc.) were introduced by electroporation. In the cloned lines, the cell strain where a wide-range sequence deletion occurs from exon 3 to exon 4 in both alleles was determined as Otx2^{-/-}ES cell. Note that, since the ES cell is established from an embryo (B6xBDF1) obtained by crossing B6 lineage mouse and BDF1 lineage mouse, which have black hair color, melanin can be synthesized. The ES cell was introduced into an embryo of a normal mouse (ICR lineage) having white hair. As a result, although transplanted cells were distributed over the whole body based on GFP expression, no accumulation of melanin was observed in the retina (FIG. 5, a bright-field image and whole body GFP image). From this, it is considered that Otx2^{-/-}ES cell has a high chimeric contribution ability to the whole body; however, it was demonstrated that Otx2^{-/-}ES cell failed to contribute to the forebrain-derived retinal pigment epithelium. Tissue sections of the forebrain region (forming the cerebrum in future) were prepared and immune-stained with an antibody (Abcam, ab18207) against beta-3 tublin, which is a neuroepithelial cell marker, and an anti-GFP antibody (Abcam, Ab13970). As a result, as shown in FIG. 5, no contribution of Otx2^{-/-}ES cell-derived cell expressing GFP to the forebrain neuroepithelium (βIII tubulin positive) was observed although the Otx2^{-/-}ES cell-derived cell expressing GFP is present at a high level in other tissues.

As shown in FIG. 5, it was found that an Otx2 homo-knockout ES cell loses a differentiation ability into a neuroepithelial cell in the forebrain region including a cell forming the cerebrum in future.

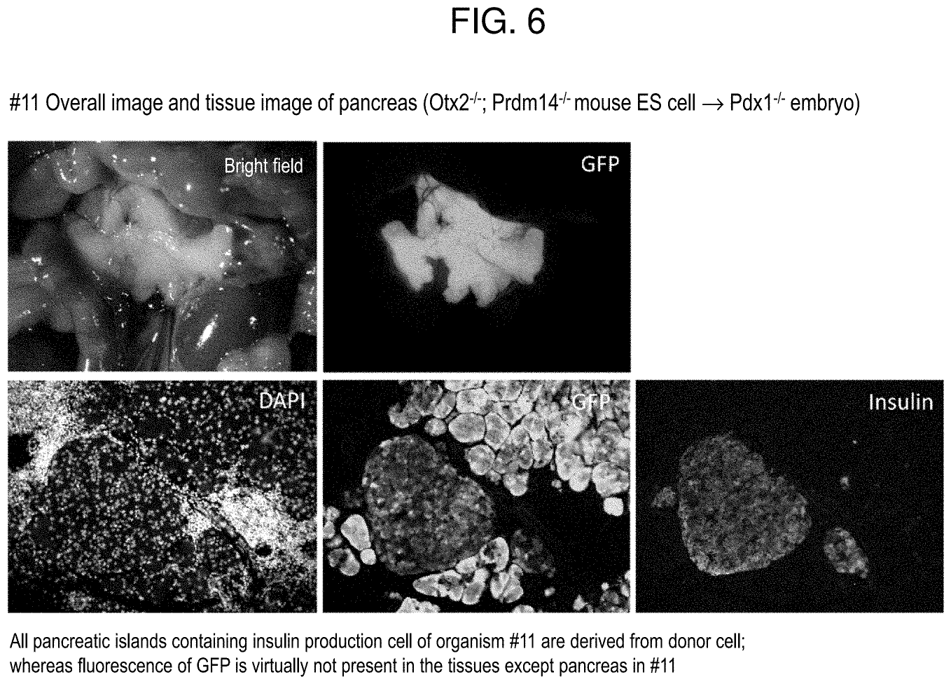

Subsequently, Prdm14 knockout and Otx2 knockout ES cells expressing GFP were introduced into Pdx1−/− knockout embryo, and then, whether the pancreas derived from the ES cells can be formed or not was checked. This is because it is known that a direct target of Otx2 gene contains a Wnt antagonist, Dkk1, which controls Wnt signal (Kimura et al., Developmental Cell 2005), and thus, it is unclear whether the Otx2 knockout cell can normally form an organ or is developed into an organ having a normal function. The results were as shown in FIG. 6. As shown in FIG. 6, in a chimeric organism (#11) of Pdx1−/− knockout embryo and a Prdm14 knockout and Otx2 knockout ES cell expressing GFP, a pancreas derived from transplanted ES cell (GFP positive) was formed. Tissue sections of the pancreas were prepared and immunostained with an anti-GFP antibody (Abcam, Ab13970) and an anti-insulin antibody (Abcam, Ab7842). As a result, it was confirmed that all pancreatic islets on tissue sections containing an insulin producing cell are derived from a transplanted cell. Since mouse #11 at the time of analysis was 10 weeks after birth, it is supported that the pancreas derived from a transplanted cell normally functions for a long term after birth. Note that, in the tissues except the pancreas tissue in mouse #11, GFP positive cells are virtually not present. The reason is considered that the chimera formation ability of the ES cell introduced in organism #11 was weak.

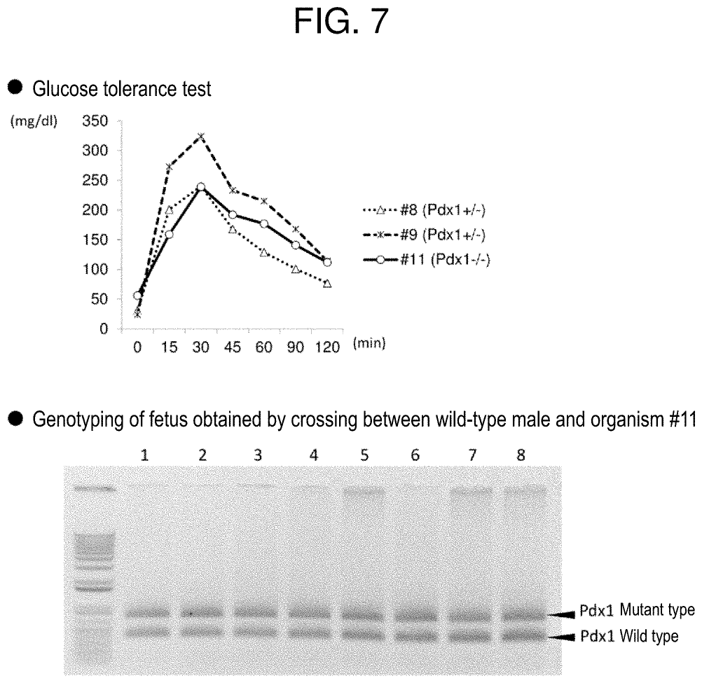

Prior to laparotomy analysis, mouse #11 and littermates (#8, #9) at 8 weeks after birth were subjected to a glucose tolerance test. The results are shown in FIG. 7. In the glucose tolerance test, a 150 mg/ml glucose/physiological saline solution was intraperitoneally injected to a mouse at a dose of 10 μL per body weight (1 g) and a change of blood-sugar level with time was measured. As the results of the glucose tolerance test, as shown in FIG. 7, it was clear that the pancreas derived from a Prdm14 knockout and Otx2 knockout ES cell formed within a Pdx1−/− knockout embryo has a normal ability to lower blood glucose and is functional.

A mouse #11 (female) was crossed with a wild type mouse to obtain F1 mice. In F1 mice (n=8), Pdx1 genotype was determined. As a result, as shown in FIG. 7, any one of F1 mice has a genotype of Pdx1+/−. This means that all the cells contributing to a germ cell had a genotype of Pdx−/−. In other words, it means that a Prdm14 knockout and Otx2 knockout ES cell (Pdx+/+) does not contribute to a germ cell. Note that, in the Pdx1 locus of the Pdx1 knockout mouse, since e.g., LacZ gene is introduced (Offield et al., Development 1996), a larger gene amplification band than that (405 bp) of the wild type is detected.

As described above, if a Prdm14 gene knockout pluripotent cell was used as the cell to be introduced into an embryo to produce a somatic chimera animal, contribution of the pluripotent stem cell to a germ cell can be reduced or deprived. Also, if an Otx2 gene knockout pluripotent cell was used as the cell to be introduced into an embryo to produce a somatic chimera animal, contribution of the pluripotent stem cell to the cerebral cortex can be reduced or deprived.

Sequence listing
SEQ ID NO: 1: Prdm14 gene targeting sequence of
gRNA
(GAACCTCGCCACCACCGAGG)
SEQ ID NO: 2: Prdm14 gene targeting sequence of
gRNA
(GTATGGAGCCATCGCTAGTC)
SEQ ID NO: 3: A Prdm14 gene sequence knocked out
(CTCGCCACCACCG GTCCGGAGCACCCAACCG)
SEQ ID NO: 4: A Prdm14 gene sequence knocked out
(CTCGCCACCACCG CAGTATTAAAACATGGATGTA)
SEQ ID NO: 5: Otx2 gene targeting sequence of
gRNA1
(GAGTCTGACCACTTCGGGTA)
SEQ ID NO: 6: Otx2 gene targeting sequence of
gRNA2
(GTATGGAGCCATCGCTAGTC)
SEQ ID NO: 7: Forward primer for Pdx1
amplification
(ATTGAGATGAGAACCGGCATG)
SEQ ID NO: 8: Reverse primer for wild type Pdx1
amplification
(TTCATGCGACGGTTTTGGAAC)
SEQ ID NO: 9: Reverse primer for mutant Pdx1
amplification
(TGTGAGCGAGTAACAACC)

1. A method for preparing a somatic chimera animal, comprising
introducing a pluripotent cell into an embryo to obtain a somatic chimera animal, wherein
the pluripotent cell is genetically engineered not to differentiate into a predetermined type of cell.

2. A composition for use in preparing a somatic chimera animal, comprising a pluripotent cell, wherein the pluripotent cell is genetically engineered not to differentiate into a predetermined type of cell.

3. The method according to claim 1, wherein the pluripotent cell genetically engineered not to differentiate into a predetermined type of cell is a cell in which a gene essential for a germ cell is disrupted.

4. The composition according to claim 2, wherein the pluripotent cell genetically engineered not to differentiate into a predetermined type of cell is a cell in which a gene essential for a germ cell is disrupted.

5. The method according to claim 1, wherein the pluripotent cell genetically engineered not to differentiate into a predetermined type of cell is a Prdm14 gene knockout cell, an Otx2 gene knockout cell or a Prdm14 gene/Otx2 gene double knockout cell.

6. The composition according to claim 2, wherein the pluripotent cell genetically engineered not to differentiate into a predetermined type of cell is a Prdm14 gene knockout cell, an Otx2 gene knockout cell or a Prdm14 gene/Otx2 gene double knockout cell.

7. The method according to claim 3, wherein the somatic chimera animal and the pluripotent cell are female.

8. The method according to claim 3, wherein the somatic chimera animal and the pluripotent cell are male.

9. (canceled)

10. A non-human somatic chimera animal, wherein a predetermined type of cell consists of a cell derived from a host animal.

11. The non-human somatic chimera animal according to claim 10, wherein a germ cell consists of the cell derived from a host animal.

12. The method according to claim 3, wherein the pluripotent cell genetically engineered not to differentiate into a predetermined type of cell is a Prdm14 gene knockout cell, an Otx2 gene knockout cell or a Prdm14 gene/Otx2 gene double knockout cell.

13. The method according to claim 5, wherein the somatic chimera animal and the pluripotent cell are female.

14. The method according to claim 5, wherein the somatic chimera animal and the pluripotent cell are male.

15. The method according to claim 12, wherein the somatic chimera animal and the pluripotent cell are female.

16. The method according to claim 12, wherein the somatic chimera animal and the pluripotent cell are male.

17. The composition according to claim 4, wherein the pluripotent cell genetically engineered not to differentiate into a predetermined type of cell is a Prdm14 gene knockout cell, an Otx2 gene knockout cell or a Prdm14 gene/Otx2 gene double knockout cell.


SEQUENCE LISTING (9 sequences)

SEQ ID NO: 1 - DNA, 20 bp, Artificial Sequence
  gRNA-1 targeting Prdm14
gaacctcgccaccaccgagg

SEQ ID NO: 2 - DNA, 20 bp, Artificial Sequence
  gRNA-2 targeting Prdm14
gtatggagccatcgctagtc

SEQ ID NO: 3 - DNA, 31 bp, Artificial Sequence
  Example of knocked out Prdm14
ctcgccaccaccggtccggagcacccaaccg

SEQ ID NO: 4 - DNA, 34 bp, Artificial Sequence
  Example 2 of knocked out Prdm14
ctcgccaccaccgcagtattaaaacatggatgta

SEQ ID NO: 5 - DNA, 20 bp, Artificial Sequence
  Otx2 targeting sequence in gRNA1
gagtctgaccacttcgggta

SEQ ID NO: 6 - DNA, 20 bp, Artificial Sequence
  Otx2 targeting sequence in gRNA2
gtatggagccatcgctagtc

SEQ ID NO: 7 - DNA, 21 bp, Artificial Sequence
  Forward primer for Pdx1 amplification
attgagatgagaaccggcatg

SEQ ID NO: 8 - DNA, 21 bp, Artificial Sequence
  Reverse primer for WT Pdx1 amplification
ttcatgcgacggttttggaac

SEQ ID NO: 9 - DNA, 18 bp, Artificial Sequence
  Reverse primer for mutant Pdx1 amplification
tgtgagcgagtaacaacc